(19) United States
(12) Patent Application Publication
(10) Pub. No.: US 2024/0312617 A1
(43) Pub. Date: Sep. 19, 2024

(54) SYSTEM AND METHOD FOR ADMINISTERING A TREATMENT AT A TREATMENT SITE IN THE BRAIN

(71) Applicant: BIONAUT LABS LTD., Herzliya (IL)

(72) Inventors: Alex KISELYOV, San Diego, CA (US); Michael SHPIGELMACHER, Los Angeles, CA (US); Michael KARDOSH, Kiryat Ono (IL); Florent CROS, Los Angeles, CA (US); Suehyun CHO, Torrance, CA (US); Darrell HARRINGTON, Canoga Park, CA (US); William G. LOUDON, San Clemente, CA (US)

(73) Assignee: BIONAUT LABS LTD., Herzliya (IL)

(21) Appl. No.: 18/671,425

(22) Filed: May 22, 2024

Related U.S. Application Data
(63) Continuation of application No. 18/242,295, filed on Sep. 5, 2023.
(60) Provisional application No. 63/404,024, filed on Sep. 6, 2022.

(51) Int. Cl. G16H 40/60; A61M 5/00

(52) U.S. Cl. CPC G16H 40/60; A61M 5/00; A61M 2210/0693

(57) ABSTRACT

A method for providing localized treatment at a target site in the brain of a patient is provided. The method comprises providing a system comprising a miniature device configured to carry one or more therapeutic components for performing the treatment, and an external system configured to direct the miniature device; inserting the miniature device to a starting location within the patient; operating the external system to direct the miniature device to travel along an access path from the starting location to the target site; and administering the therapeutic component at the target site. A system for performing the method is further provided.

### TECHNOLOGICAL FIELD

The presently disclosed subject matter relates to systems and miniature device configured to navigate within a patient to perform a therapeutic activity at a predetermined treatment site in the brain, and in particular to such systems which use magnetic fields to direct operation of miniature devices within the patient.

### BACKGROUND

Brainstem glioma (BSG) is a disease in which a tumor forms on glial cells of the brainstem of a patient. A common form of BSG is a diffuse intrinsic pontine glioma, which forms on the pons of the brainstem. This area of the brain, in particular the ventral side thereof, is difficult to reach by conventional methods, complicating efforts to deliver therapeutic compounds directly to the site of a BSG.

### SUMMARY

According to an aspect of the presently disclosed subject matter there is provided a method for providing localized treatment at a target site in the brain of a patient, the method comprising:
providing a system comprising a miniature device configured to carry one or more therapeutic components for performing the treatment, and an external system configured to direct the miniature device;
inserting the miniature device to a starting location within the patient;
operating the external system to direct the miniature device to travel along an access path from the starting location to the target site; and
administering the therapeutic component at the target site.

The starting location may be inside a posterior area of the skull substantially opposite the pons, for example located substantially along the median plane of the patient, or up to about 45° from the median plane of the patient. The access path may pass through the parenchyma. The access path may pass the cerebellum, and pass one or more cerebellar peduncles. The miniature device may be introduced to the starting point via a burr hole formed in the patient's skull.

The starting location may be in the parenchyma adjacent the cisterna magna of the patient. The access path may comprise a substantially curvilinear portion. The access path may pass through the parenchyma. The access path may pass the cerebellum, and pass one or more cerebellar peduncles or near the cerebellar peduncles.

The starting location may be in the subarachnoid space of the patient, for example in the cisterna magna. The access path may comprise a substantially curvilinear portion. The access path may pass through the cerebrospinal fluid and the parenchyma. The access path may pass through the cisterna magna, pass the cerebellum, and pass one or more cerebellar peduncles or near the cerebellar peduncles. The access path may pass through the pia mater.

The starting location may be in the subdural space of the patient, for example in the spinal cord. The access path may comprise a substantially curvilinear portion. The access path may pass through the cerebrospinal fluid and the parenchyma. The access path may pass through the subdural space of the spinal cord, pass through the foramen magnum, pass through the cisterna magna, pass the cerebellum, and pass one or more cerebellar peduncles or near the cerebellar peduncles. The access path may further pass through the pia mater.

The access path may pass through the subdural space of the spinal cord, pass through the foramen magnum, pass through the cisterna magna, pass through the foramen of Magendie, and pass through the fourth ventricle. The access path may further pass through the aqueduct of Silvius, and through the third ventricle. The access path may further pass through the pia mater.

The target site may be in the cerebral cortex, for example in the frontal lobe.

The target site may be in the brain stem. According to some examples, the target site is in the pons. According to some examples, the target site is in the midbrain.

The method may further comprise directing the miniature device to travel along a return path after the therapeutic component has been administered at the target site.

The return path may be substantially the same as the access path.

The localized treatment may be directed towards treatment of a brainstem glioma. The brainstem glioma may be a diffuse intrinsic pontine glioma.

According to another aspect of the presently disclosed subject matter, there is provided a system configured for performing the above-described method.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a horizontal cross-sectional view of a patient's brain, illustrating a path to a treatment site according to an example of the presently disclosed subject matter;

FIG. 1B is a partial side-posterior view of a patient's brain, showing the path illustrated in FIG. 1A;

FIG. 2A is a sagittal cross-sectional view of a patient's brain, illustrating a path to a treatment site according to another example of the presently disclosed subject matter;

FIG. 2B is a partial side-posterior view of a patient's brain, showing the path illustrated in FIG. 2A; and

FIGS. 3 and 4 are sagittal cross-sectional views of patients' brains, illustrating paths to treatment sites according to other examples of the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In one aspect, provided herein are devices and systems that comprise a millimeter-scale tetherless object controlled or manipulated remotely by an external magnetic field, referred to herein as the “miniature device,” and separate from the miniature device an interactive hardware-software platform, referred to herein as the “external system,” that generates, modulates and controls magnetic fields in a defined three-dimensional operational volume to propel and navigate the miniature device to a specific anatomical target to complete a mission. In particular, a system may be provided according to the presently disclosed subject matter, comprising a suitable external system and one or more suitable miniature devices. An example of such a system is described in PCT application PCT/US2022/40303, the full contents of which are incorporated herein by reference.

The miniature device is propelled and navigated remotely by the external system through specific anatomical milieu, exemplified by the lumen, cavity, vessel, tissue(s), and circuitry. The milieu can include homogeneous or heterogeneous components, such as lumens, respective luminal lining, and adjacent tissue(s). One such heterogeneous compartment is the central nervous system (CNS).

The miniature device may be navigated through healthy and/or pathological domains to a target site to administer a treatment to treat diseased tissue at or near the target site. According to some examples, the diseased tissue is a brainstem glioma (BSG), for example comprising a malignancy. The BSG may be a diffuse intrinsic pontine glioma or a focal glioma, for example located in the pons part of the brainstem. Administering the treatment may comprise delivery of a therapeutic component, for example comprising one or more chemical compounds, one or more small molecules, biologics, cells, one or more radioisotopes, etc., for treating the diseased tissue.

A microsurgical, diagnostic, or therapeutic mission comprises one or more actions, to be accomplished in the relevant anatomical milieu.

### The Mission

The mission for administering a treatment for a BSG may comprise:
(a) travel of the miniature device to the target site inside the milieu as described herein;
(b) administering the treatment to the diseased tissue at the target site; and
(c) optionally travel of the miniature device to exit the patient.

The mission may comprise each of several miniature devices traveling to the target site to administer a treatment, the same miniature device traveling to several target sites, the miniature device administering several treatments at the target site, the miniature device repeatedly administering a single therapeutic component or administering different therapeutic components at one or more target sites at different times, etc. For clarity of disclosure, examples of a single miniature device traveling to a single treatment site to administer a single therapeutic component will be described as an example only, and missions of other parameters, e.g., as described above in this paragraph, are included in such examples implicitly, mutatis mutandis.

Travel of the miniature device within the patient requires control and a set of specific actions mediated by an external system described herein. In some embodiments, travel is performed in a first volume filled with media or liquid such as, but not limited to, cerebrospinal fluid (CSF).

According to some examples, the path traveled by the miniature device is linear. In other embodiments, the three-dimensional path traveled by the miniature device is curvilinear.

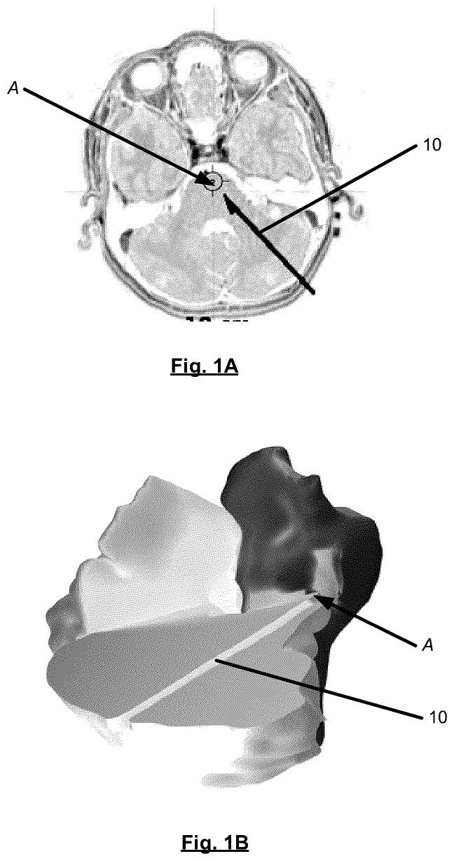

According to some examples, for example as illustrated in FIGS. 1A and 1B, travel of the miniature device to the target site comprises forming a burr hole in a posterior area of the skull, for example opposite or slightly above it. The burr hole may be in the center of the skull, i.e., located substantially on the median plane, or to one side, for example located up to about 45° from the median plane. The miniature device may be inserted directly into the parenchyma at a starting location before being directed by the external system along a path of travel, for example as indicated at 10 in FIGS. 1A and 1B, to the treatment site, indicated at A. The path of travel may be substantially linear from the burr hole to the target site at or adjacent the pons. The path may be substantially similar to one which is conventionally used when performing a posterior fossa biopsy, for example to access a pontine lesion. According to some examples, the miniature device travels through the parenchyma, traversing the cerebellum, one or more cerebellar peduncles, and ultimately the pons. It will be appreciated that while a substantially linear path is described, in practice the miniature device may be diverted from the path as necessary, for example in view of the specific anatomy of the patient.

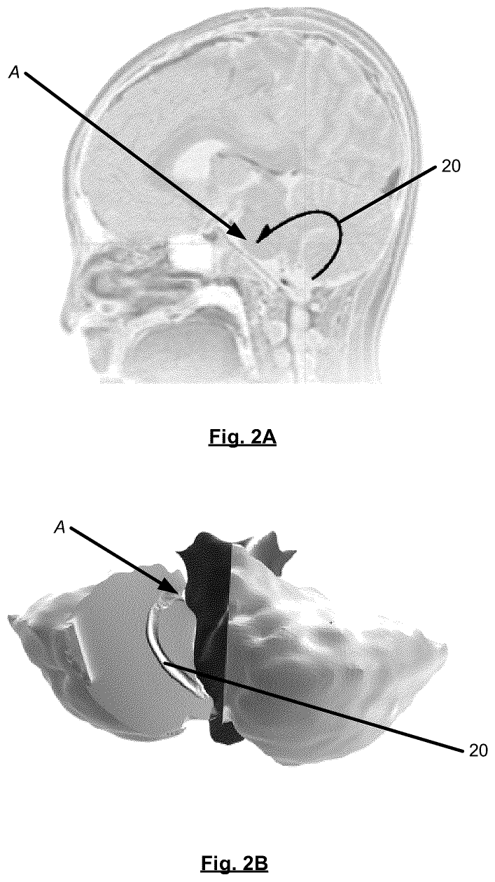

According to other examples, for example as illustrated in FIGS. 2A and 2B, travel of the miniature device to the target site comprises insertion of the miniature device to the cisterna magna of the patient by any suitable method, e.g., using a cannula, for example as is well known in the art. A burr hole may be used if necessary. The miniature device may then be inserted, e.g., by one performing the mission, into the parenchyma at a starting location. The external system is then used to direct the miniature device to move along a substantially curvilinear path of travel, for example as indicated at 20 in FIGS. 2A and 2B, to the treatment site, indicated at A. According to some examples, the miniature device travels through the parenchyma, traversing the cerebellum, one or more cerebellar peduncles or near the cerebellar peduncles, and ultimately the pons.

According to other examples, for example as illustrated in FIG. 3, travel of the miniature device to the target site comprises insertion of the miniature device into the subarachnoid space of the patient by any suitable method, e.g., using a cannula, for example as is well known in the art. A burr hole may be used if necessary. The miniature device may be inserted into the cisterna magna, into the spinal cord, or in any other suitable starting location.

According to some examples, the miniature device may be inserted into the cisterna magna, and the external system is then used to direct the miniature device to move along a substantially curvilinear path of travel, for example as indicated at 30a in FIG. 3, to the treatment site, indicated at A. According to these examples, the miniature device traverses the cisterna magna, the cerebellum, one or more cerebellar peduncles or near the cerebellar peduncles, and ultimately the pons. The route is through CSF (indicated by dotted lines), and entering the parenchyma (indicated by solid lines) via the pia mater, for example at or near the treatment site at the pons.

According to some examples, the miniature device may be inserted into the spinal cord, for example in the subdural space thereof. This may be done at any suitable location, including, but not limited to, the lumbar region. The external system is then used to direct the miniature device to move along a substantially curvilinear path of travel, for example as indicated at 30b in FIG. 3, to the treatment site, indicated at A. According to these examples, the miniature device travels from the subdural space of the spinal cord through the foramen magnum, traversing the cisterna magna, the cerebellum, one or more cerebellar peduncles or near the cerebellar peduncles, and ultimately the pons. The route is through CSF (indicated by dotted lines), and entering the parenchyma (indicated by solid lines) via the pia mater, for example at or near the treatment site at the pons.

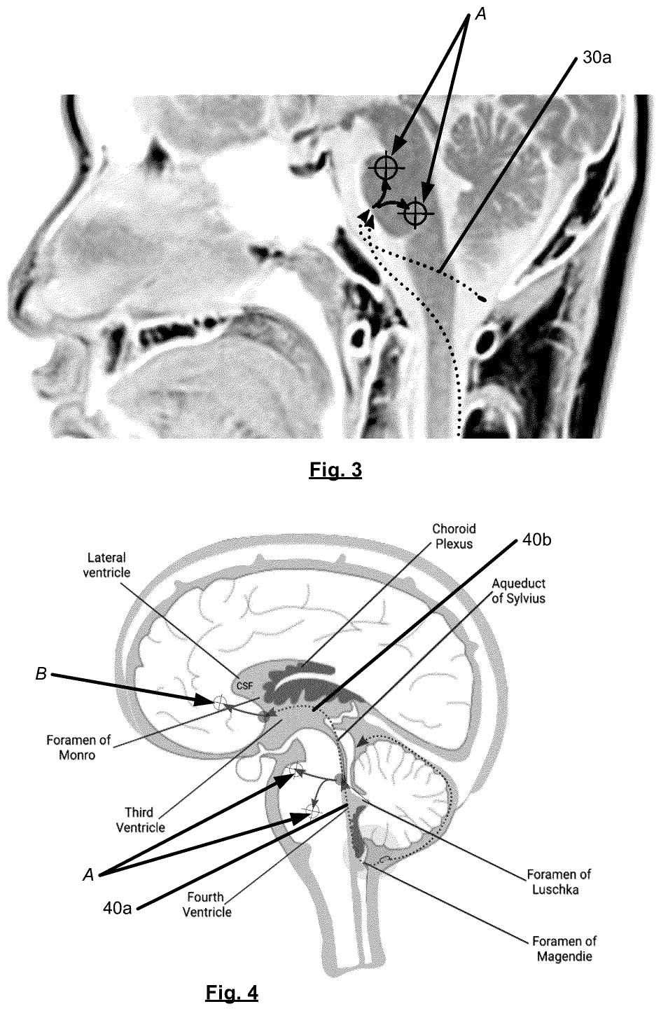

According to other examples, for example as illustrated in FIG. 4, travel of the miniature device to the target site comprises insertion of the miniature device into the subarachnoid space of the patient by any suitable method, e.g., using a cannula, for example as is well known in the art. A burr hole may be used if necessary. The miniature device may be inserted into the spinal cord at any suitable location, including, but not limited to, the lumbar region.

According to some examples, the external system is then used to direct the miniature device to move along a substantially curvilinear path of travel, for example as indicated at 40a in FIG. 4, to a treatment site, indicated at A. According to these examples, the miniature device travels from the subdural space of the spinal cord through the foramen magnum, traversing the cisterna magna, passing through the foramen of Magendie into the fourth ventricle, and ultimately the pons. The route is through CSF (indicated by dotted lines), and entering the parenchyma (indicated by solid lines) via the pia mater, for example at or near the treatment site.

According to other examples, the external system is then used to direct the miniature device to move along a substantially curvilinear path of travel, for example as indicated at 40b in FIG. 4. According to these examples, the miniature device travels from the subdural space of the spinal cord through the foramen magnum, traversing the cisterna magna, passing through the foramen of Magendie into the fourth ventricle, passing through the aqueduct of Silvius to the third ventricle, and ultimately reaching a treatment site beyond the pons, for example in the midbrain, in an area of the cerebral cortex such as the frontal lobe (as indicated at B), etc. The route is through CSF (indicated by dotted lines), and entering the parenchyma (indicated by solid lines) via the pia mater, for example at or near the treatment site.

According to any of the above examples, the miniature device may be directed to travel so as to exit the patient, for example along substantially the same route it traveled to reach the treatment site.

### The External System

The external system may be provided as described in PCT application PCT/US2022/40303. According to some examples, it may comprise a software module and a hardware system. The external system uses magnetic fields to exert mechanical forces on the miniature device and to control it to perform specific actions. The forces, controllably and predictably exerted on the miniature device, are expected to have various consequences on the milieu and on the path thereto. In some embodiments, the external system mediates propulsion and navigation of the miniature device from one predetermined position to another. For example, the external system controls specific miniature device motion(s) including, but not limited to, standalone axial or diametral spinning, rotation, vibration, tumbling, crawling, rocking, lateral motion, etc. In addition, according to some examples the external system is configured to direct the miniature device to release a therapeutic component.

In some embodiments, the external system generates magnetic fields using permanent magnets. In some embodiments, the permanent magnets are mounted on a mechanical setup that holds the magnet and can move in three dimensions. In some embodiments, the permanent magnets can rotate. In some embodiments, there is only one permanent magnet. In other embodiments, there are several magnets that are actuated independently.

In some embodiments, the external system generates magnetic fields using electromagnets. The electromagnet may comprise one or more coils. An electromagnet can, in addition, comprise a bobbin to support the coil and a yoke that modifies the electromagnetic properties of the coil. In some embodiments, the electromagnets are mounted on a mechanical setup that holds them at a fixed location with respect to the milieu where the miniature device resides. In some embodiments, the electromagnets are mounted on a mechanical setup that controllably and predictably determines the position of the electromagnets with respect to the milieu where the miniature device is located. In some embodiments, the electromagnets are actuated according to predetermined control algorithms that take into account the position, velocity, acceleration, and orientation of the miniature device, for example determined based on X-ray images of the miniature device in real time. In some embodiments, the magnetic fields are generated in such a fashion that the forces applied to the miniature device cause it to rotate about its axis.

In some embodiments, the external system generates magnetic fields using a combination of electromagnets and permanent magnets. In some embodiments, both electromagnets and permanent magnets are used in concert to generate the fields and applies forces to the miniature device.

In some embodiments, the external system generates the magnetic fields using six to twelve electromagnetic coils. For example, eight electromagnetic coils are used to generate the magnetic fields. Each coil is between 4″×4″×4″ and 12″×12″×24″ in size, for example, each electromagnetic coil is 8″×8″×15″ in size. Each coil can carry up to 100 amps, for example running at 30 amps. In some embodiments, the electromagnetic coils have a ferromagnetic yoke. In other embodiments, the electromagnetic coils have no yoke. In some embodiments, the electromagnetic coils are arranged around the head of the subject in use. In some embodiments, the electromagnetic coils are between 5 cm to 25 cm, e.g., 15 cm, from the milieu.

The external system may use a variety of visualization or imaging systems to assist with the application of the forces and result in adequate control of the miniature device. In some embodiments, the external system uses X-rays to image the miniature device inside the targeted milieu. In some embodiments, the external system uses two X-rays applied in a stercovision system to image the miniature device inside the targeted milieu to determine its three-dimensional position with respect to landmarks or fiducial markers used as a reference. In some embodiments, the external system uses optical stercovision to determine the position of the electromagnets or permanent magnets with respect to fiducial markers or landmarks used as a reference. In some embodiments, the external system uses a combination of X-ray stercovision and optical stereovision to monitor the position of the miniature device, only visible under X-ray vision, with respect to the external electromagnets or permanent magnets, only visible under optical vision.

The external system software module comprises: a planning software submodule and a hardware-control software submodule. In some embodiments, the external system software module makes use of pre-recorded digital data, such as MRI scans, CT scans, etc. . . . to assist with the activities performed by both submodules. In some embodiments, MRI scans are analyzed by the software module and digital three-dimensional objects representing various tissue masses present in the host environment are generated automatically. In some embodiments, the hardware-control software module uses digital information from the optical or X-ray stereovision system and computes automatically the mathematical transformation to allow display, in a single referential, three-dimensional data generated from MRI, vision and X-ray system.

### The Miniature Device

The miniature device may be provided as described in PCT application PCT/US2022/40303. The miniature device's dimensions, geometry, etc., are selected to facilitate performance of its mission within the milieu.

In some embodiments, the miniature device is elongated in one dimension. In some embodiments, the miniature device has a total length between about 1 mm and about 20 mm, for example about 7 mm. In some embodiments, the miniature device has an outer diameter between about 1 mm and about 5 mm, for example about 2.5 mm. In some embodiments, the miniature device has a total length between about 50 μm and about 10,000 μm. In some embodiments, the miniature device has an outer diameter between about 50 μm and about 50,000 μm.

The miniature device comprises a body having any suitable shape, including, but not limited to, a cylinder, a cone, a trapezoid, a sphere, or a spheroid. In some embodiments, the body is made of or comprises a permanent magnet magnetized along a longitudinal axis of the miniature device. In some embodiments, the permanent magnet is magnetized along a direction that is substantially perpendicular to a longitudinal axis of the miniature device. In some embodiments, the body is made of a radiopaque material. In some embodiments, the body is made of a rare earth magnet, coated with electroplated nickel and gold, ceramic, plastic, or other substantially non-magnetic biocompatible material.

In some embodiments, the body of the miniature device is made from a neodymium magnet. In some embodiments, the grade of the neodymium magnet is from N40 to N55, e.g., N50. In some embodiments, the magnet's residual induction (Bᵣ) is between about 12 kG and about 15 kG, e.g., about 14 kG. In some embodiments, (BH)ₘₐₓ is between about 38 MGOe and about 56 MGOe, e.g., about 47 MGOe. In some embodiments, the magnet's intrinsic coercivity, (Hci) is greater than about 11 kOe. In some embodiments, the magnet's intrinsic normal coercivity, (HcB) is greater than about 10 kOc. In some embodiments, the magnet's maximum operating temperature is about 60° C. to about 80° C., preferably about 80° C.

In some embodiments, the miniature device is configured to be driven by a static unidirectional force, for example arising from the interaction between the magnetic field generated by the external system and a permanent magnet of the miniature device.

### The Introduction/Retrieval Kit

The system may comprise an introduction/retrieval kit may be provided, for example as described in PCT application PCT/US2022/40303, the full contents of which are incorporated herein by reference. The introduction/retrieval kit comprises a sharp rigid pointed surgical instrument fitted with a cannula. In some embodiments, the cannula is rigid and made of titanium or other non-magnetic metal or plastic. In some embodiments, the cannula is flexible. In some embodiments, the cannula outer diameter ranges from about 1 mm to about 10 mm, for example about 3.5 mm. In some embodiments, the cannula is stabilized by a mechanical arm. In some embodiments, the cannula is designed to be stabilized by a user. In some embodiments, the cannula is configured to be guided using X-rays. In some embodiments, the cannula is configured to be guided using a stereotactic technique.

In some embodiments, the introduction/retrieval kit further comprises an introducer comprising a separate, interchangeable miniature device holder configured to insert the miniature device into the subject and to release it into, e.g., the cisterna magna. In some embodiments, the introducer is disposable and/or configured for a single use. In some embodiments, the introducer is pre-loaded at its distal end with the miniature device. The miniature device may be held in place by a small, e.g., having a diameter of 0.75 mm, magnet at the distal end of the introducer. The small magnet may be disposed at the bottom of the introducer, thereby holding the miniature device in place, and facilitating controlled orientation of it before introduction of the miniature device into the patient.

In some embodiments, the introduction/retrieval kit further comprises a separate, interchangeable miniature retriever, which may replace the introducer in the cannula and is used to retrieve the miniature device from the patient, for example once the mission is complete. In some embodiments, the retriever is disposable and/or configured for a single use. In some embodiments, the retriever has a tip with a magnet at its distal end that is used to attract and capture the miniature device and remove it from the subject. The tip of the retriever may comprise a cage having a small, e.g., no greater than about 2 mm, spherical magnet that is configured to freely rotate, thereby facilitating controlling the orientation of the miniature device during retrieval from the patient.

In some embodiments, a single mechanism constitutes the introducer and the retriever. In some embodiments, the retriever is a separate tool that comprises a net to catch the miniature device and a magnet at the end of a wire to attract the miniature device towards the net. In some embodiments, the external system drives the miniature device back to the net and the net is mechanically actuated using tethers to close around the miniature device.

In some embodiments, a sharp rigid pointed surgical instrument fitted with a cannula enters the body in the neck area and is pushed through the soft tissue. In some embodiments, the tip of the sharp rigid pointed surgical instrument is fitted with a cannula and advanced until it reaches the cisterna magna.

In some embodiments, the external system monitors the position of the sharp rigid pointed surgical instrument fitted with a cannula using a stereo vision camera. In some embodiments, the route of the sharp rigid pointed surgical instrument fitted with a cannula is planned in advance by medical personnel using the planning submodule. In some embodiments, the route is charted in the planning submodule using MRI data. In some embodiments, the position of the sharp rigid pointed surgical instrument fitted with a cannula is represented, as perceived by an optical stercovision system, in real time, on an electronic display station.

In some embodiments, the proximal end of the cannula is equipped with a valve-like system through which the sharp rigid instrument can be removed while preventing fluids from inside the body to drain out. In some embodiments, the sharp instrument is removed to make way for the miniature device to travel up and down the cannula. In some embodiments, the miniature device is placed inside the cannula, through the valve-like system and coaxed to travel all the way to the tip of the cannula by flushing a fluid. In some embodiments, a miniature device holder that fits inside the cannula and that holds the miniature device on its distal end is inserted into the cannula. In some embodiments, the miniature device holder is longer than the cannula. In some embodiments, the miniature device holder distal end is advanced further, until the miniature device is removed fully from the cannula. In some embodiments, the miniature device holder can be coaxed to release its hold on the miniature device on demand using mechanical levers or release wires available on the proximal end.

In some embodiments, the action of releasing the hold on the miniature device is monitored by the external system. In some embodiments, the action of releasing the hold on the miniature device is synchronized with the external system. In some embodiments, the external system uses stereo X-ray vision system to evaluate the position of the miniature device. In some embodiments, the external system starts generating magnetic fields of adequate intensity and characteristic as the miniature device is being released from the miniature device holder.

One or more components of the system may be provided, mutatis mutandis, as described in any one or more of WO 2019/213368, WO 2019/213362, WO 2019/213389, WO 2020/014420, WO 2020/092781, WO 2020/092750, WO 2018/204687, WO 2018/222339, WO 2018/222340, WO 2019/212594, WO 2019/213368, WO 2019/005293, WO 2020/096855, WO 2020/252033, WO 2021/021800, WO 2021/092076, WO 2021/126905, WO 2021/216463, WO 2022/119816, and U.S. Provisional application Nos. 63/191,454, 63/191,418, 63/191,515, and 63/191,497, the full contents of which are incorporated herein by reference.

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1.-36. (canceled)

37. A method for treating a pathological process within a parenchyma of a brain of an individual, comprising:
(a) delivering a tetherless miniature device to a location that is proximal to an outer surface of the parenchyma, wherein the tetherless miniature device is configured to be controlled through use of a magnetic field that is located externally to the individual;
(b) driving the tetherless miniature device into the parenchyma; and
(c) directing, using the magnetic field, the tetherless miniature device through a curvilinear path of travel within the parenchyma.

38. The method of claim 37, wherein the pathological process is a condition of the brainstem of the individual.

39. The method of claim 37, wherein the tetherless miniature device is delivered to the location that is proximal to the outer surface of the parenchyma from a spinal column of the individual.

40. The method of claim 37, wherein the tetherless miniature device is delivered to the location that is proximal to the outer surface of the parenchyma from a burr hole in the skull of the individual.

41. The method of claim 37, wherein the pathological process is a glioma.

42. The method of claim 37, wherein a length of the tetherless miniature device is no greater than 20 mm.

43. The method of claim 37, wherein the tetherless miniature device comprises a magnet.

44. The method of claim 37, wherein the location that is proximal to the outer surface of the parenchyma is within a subdural space or a subarachnoid space.

45. The method of claim 37, wherein the magnetic field is generated by a plurality of adjustable magnets.

46. The method of claim 37, wherein the tetherless miniature device moves in response to a modulation in the magnetic field.

47. The method of claim 37, wherein the curvilinear path of travel within the parenchyma passes through a cerebellum of the individual.

48. The method of claim 37, wherein the curvilinear path of travel within the parenchyma passes through a pons of the individual.

49. The method of claim 37, comprising (d) delivering a therapeutic agent, a diagnostic agent, or a combination thereof to a location of the pathological process.

50. The method of claim 49, wherein the therapeutic agent is delivered to a second location.

51. The method of claim 49, wherein the therapeutic agent is delivered along the curvilinear path of travel.

52. The method of claim 51, wherein the therapeutic agent comprises a chemical compound.

53. The method of claim 52, wherein the chemical compound comprises a small molecule or a biologic.

54. The method of claim 51, therein the therapeutic agent comprises a cell.

55. The method of claim 51, wherein the therapeutic agent comprises a radioisotope.

56. The method of claim 37, wherein the tetherless miniature device travels along a substantially linear path through at least a portion of the cerebellum before it is directed through the curvilinear path of travel within the parenchyma.
